(19) United States
(12) Patent Application Publication
(10) Pub. No.: US 2022/0047534 A1
(43) Pub. Date: Feb. 17, 2022

(54) METHODS OF MAKING AND USING PHYTOCANNABINOIDS COMPLEXED WITH A PROTEIN, PEPTIDE, AMINO ACID, POLYSACCHARIDE, DISACCHARIDE, OR MONOSACCHARIDE

(71) Applicant: John Chancey, Edmond, OK (US)

(72) Inventor: John Chancey, Edmond, OK (US)

(21) Appl. No.: 17/513,184

(22) Filed: Oct. 28, 2021

Related U.S. Application Data
(63) Continuation-in-part of application No. 16/787,460, filed on Feb. 11, 2020.
(60) Provisional application No. 62/803,694, filed on Feb. 11, 2019.

(51) Int. Cl. A61K 31/19; A61K 47/64; A61K 47/54; A61K 47/61; A23L 33/11; A23L 33/175; A23L 33/185; A23L 33/19

(52) U.S. Cl. CPC A61K 31/19; A61K 47/64; A61K 47/542; A61K 47/549; A23V 2002/00; A23L 33/11; A23L 33/175; A23L 33/185; A23L 33/19; A61K 47/61

(57) ABSTRACT

The present invention provides a phytochemical complex comprising a therapeutically effective amount of boswellic acid; and one or more complexing agents conjugated to the therapeutically effective amount of boswellic acid, wherein the one or more complexing agents are selected from proteins, peptides, amino acids, polysaccharides, disaccharides, monosaccharides, amino sugars, glycosaminoglycans, and glycol-proteins, disposed in a pharmaceutically acceptable excipient, diluent, or carrier.

### CROSS-REFERENCE TO RELATED APPLICATIONS

This application is a Continuation-In-Part of U.S. patent application Ser. No. 16/787,460 filed Feb. 11, 2020, which claims priority to U.S. Provisional Application No. 62/803,694 filed Feb. 11, 2019. The contents of each of which is incorporated by reference in their entireties.

### TECHNICAL FIELD OF THE INVENTION

The present invention is in the field of formulations of nutraceuticals, and more specifically, of a phytochemical complexed with a protein, peptide, amino acid, polysaccharide, disaccharide, or monosaccharide used in nutraceuticals.

### STATEMENT OF FEDERALLY FUNDED RESEARCH

None.

### INCORPORATION-BY-REFERENCE OF MATERIAL FILED ON COMPACT DISC

None.

### BACKGROUND ART

Without limiting the scope of the invention, its background is described in connection with a phytochemical (polyphenols, terpenoids, phytosterols, and or alkaloids) complexed with a protein, peptide, amino acid, polysaccharide, disaccharide, or monosaccharide used in nutraceuticals. Phytochemicals include, but not limited to, phenolic acids (e.g. curcumin), flavonols (e.g. quercetin), stilbenes (e.g. resveratrol), phytosterols (e.g. β-sitosterol found in Serenoa repens, Saw Palmetto and ergosterol found in fungi), lignins, carotenoids, anthocyanidins, and tannins Terpenoids include, but not limited to, monoterpenes (e.g. harpagoside found in Harpagophytum procumbens, Devil's Claw), diterpenes, triterpenes, tetracyclic triterpenes, pentacyclic triterpenes (e.g. boswellic acid found in Boswellia serrata, Boswellia and betulinic acid found in Inonotus obliquus, Chaga) Today there is a growing public awareness for healthy nourishment that includes daily amounts of required micronutrients such as vitamins, essential fatty acids and antioxidants. One source of this healthy nourishment is nutraceuticals.

### DISCLOSURE OF THE INVENTION

In the present invention a phytochemical complexes with proteins, peptides, amino acids, polysaccharides, disaccharides, monosaccharides, amino sugars, glycosaminoglycans, glycol-proteins. Also disclosed are methods of preparing a phytochemical complex, comprising obtaining a phytochemical; obtaining a protein; and mixing the phytochemical and the protein in a solvent. Also disclosed are methods of treating a subject, the method comprising identifying a subject in need of treatment of a phytochemical-related disorder, and administering to the subject a nutraceutical composition comprising a phytochemical-protein complex as described. Also disclosed are methods of preparing a phytochemical complex, comprising obtaining a phytochemical; obtaining a peptide; and mixing the phytochemical and the peptide in a solvent. Also disclosed are methods of treating a subject, the method comprising identifying a subject in need of treatment of a phytochemical-related disorder, and administering to the subject a nutraceutical composition comprising a phytochemical-peptide complex as described. Also disclosed are methods of preparing a phytochemical complex, comprising obtaining a phytochemical; obtaining an amino acid; and mixing the phytochemical and the amino acid in a solvent. Also disclosed are methods of treating a subject, the method comprising identifying a subject in need of treatment of a phytochemical-related disorder, and administering to the subject a nutraceutical composition comprising a phytochemical-amino acid complex as described. Also disclosed are methods of preparing a phytochemical complex, comprising obtaining a phytochemical; obtaining a polysaccharide; and mixing the phytochemical and the polysaccharide in a solvent. Also disclosed are methods of treating a subject, the method comprising identifying a subject in need of treatment of a phytochemical-related disorder, and administering to the subject a nutraceutical composition comprising a phytochemical-polysaccharide complex as described. Also disclosed are methods of preparing a phytochemical complex, comprising obtaining a phytochemical; obtaining a disaccharide; and mixing the phytochemical and the disaccharide in a solvent. Also disclosed are methods of treating a subject, the method comprising identifying a subject in need of treatment of a phytochemical-related disorder, and administering to the subject a nutraceutical composition comprising a phytochemical-disaccharide complex as described. Also disclosed are methods of preparing a phytochemical complex, comprising obtaining a phytochemical; obtaining a monosaccharide; and mixing the phytochemical and the monosaccharide in a solvent. Also disclosed are methods of treating a subject, the method comprising identifying a subject in need of treatment of a phytochemical-related disorder, and administering to the subject a nutraceutical composition comprising a phytochemical-monosaccharide complex as described. Also disclosed are nutraceutical compositions comprising a phytochemical complexed with a protein, peptide, amino acid, polysaccharide, disaccharide, or monosaccharide as described and a pharmaceutically acceptable excipient, diluent, or carrier.

The therapeutically effective amount of one or more phytochemicals may be non-covalently conjugated to the complexing agent. The therapeutically effective amount of one or more phytochemicals may be 2, 3, 4, 5, 6, or more phytochemicals. The proteins may be selected from Whey protein isolate, Egg protein isolate, Oat protein isolate, Hemp protein, Sunflower protein isolate, Pea protein isolate, soybean protein isolate, fishmeal, flaxseed and Brown rice protein isolate. The one or more complexing agents may be N-acetylglucosamine, glucosamine sulfate or N-acetylgalactosamine, glucuronic acid, iduronic acid, galactose chondroitin and glucosamine, glycosaminoglycan. The one or more complexing agents may be Cysteine, N-Acetyl cysteine, Methionine, DL methionine, L methionine, Tyrosine, taurine. The one or more complexing agents may be Glutathione. The therapeutically effective amount of one or more terpenes with complexing agents selected from whey protein isolate, egg protein isolate, oat protein isolate, hemp protein, sunflower protein isolate pea protein isolate, soybean protein isolate, fishmeal, flaxseed, brown rice protein isolate, N-acetylglucosamine, glucosamine sulfate or N-acetylgalactosamine, glucuronic acid, iduronic acid, galactose chondroitin and glucosamine, glycosaminoglycan, Cysteine, N-Acetyl cysteine, Methionine, DL methionine, L methionine, Tyrosine, taurine, Glycose aminoglycans, mucopolysaccharides, polysaccharide, chondroitin, Chondroitin sulfate, glucosamine, Glucosamine sulfate, carrageenan, ulvan, fucoidan, or a combination thereof. The therapeutically effective amount of one or more phytochemicals comprise a monoterpene and the one or more complexing agents are selected from whey protein isolate, egg protein isolate, oat protein isolate, hemp protein, sunflower protein isolate pea protein isolate, soybean protein isolate, fishmeal, flaxseed, brown rice protein isolate, N-acetylglucosamine, glucosamine sulfate or N-acetylgalactosamine, glucuronic acid, iduronic acid, galactose chondroitin and glucosamine, glycosaminoglycan, Cysteine, N-Acetyl cysteine, Methionine, DL methionine, L methionine, Tyrosine, taurine, Glycose aminoglycans, mucopolysaccharides, polysaccharide, chondroitin, Chondroitin sulfate, glucosamine, Glucosamine sulfate, carrageenan, ulvan, fucoidan, Glutathione, or a combination thereof. The therapeutically effective amount of one or more phytochemicals comprise a diterpene and the one or more complexing agents are selected from whey protein isolate, egg protein isolate, oat protein isolate, hemp protein, sunflower protein isolate pea protein isolate, soybean protein isolate, fishmeal, flaxseed, brown rice protein isolate, N-acetylglucosamine, glucosamine sulfate or N-acetylgalactosamine, glucuronic acid, iduronic acid, galactose chondroitin and glucosamine, glycosaminoglycan, Cysteine, N-Acetyl cysteine, Methionine, DL methionine, L methionine, Tyrosine, taurine, Glycose aminoglycans, mucopolysaccharides, polysaccharide, chondroitin, Chondroitin sulfate, glucosamine, Glucosamine sulfate, carrageenan, ulvan, fucoidan, Glutathione, or a combination thereof. The therapeutically effective amount of one or more phytochemicals comprise a triterpene and the one or more complexing agents are selected from whey protein isolate, egg protein isolate, oat protein isolate, hemp protein, sunflower protein isolate pea protein isolate, soybean protein isolate, fishmeal, flaxseed, brown rice protein isolate, N-acetylglucosamine, glucosamine sulfate or N-acetylgalactosamine, glucuronic acid, iduronic acid, galactose chondroitin and glucosamine, glycosaminoglycan, Cysteine, N-Acetyl cysteine, Methionine, DL methionine, L methionine, Tyrosine, taurine, Glycose aminoglycans, mucopolysaccharides, polysaccharide, chondroitin, Chondroitin sulfate, glucosamine, Glucosamine sulfate, carrageenan, ulvan, fucoidan, Glutathione, or a combination thereof. The therapeutically effective amount of one or more phytochemicals comprise tetracyclic triterpene and the one or more complexing agents are selected from whey protein isolate, egg protein isolate, oat protein isolate, hemp protein, sunflower protein isolate pea protein isolate, soybean protein isolate, fishmeal, flaxseed, brown rice protein isolate, N-acetylglucosamine, glucosamine sulfate or N-acetylgalactosamine, glucuronic acid, iduronic acid, galactose chondroitin and glucosamine, glycosaminoglycan, Cysteine, N-Acetyl cysteine, Methionine, DL methionine, L methionine, Tyrosine, taurine, Glycose aminoglycans, mucopolysaccharides, polysaccharide, chondroitin, Chondroitin sulfate, glucosamine, Glucosamine sulfate, carrageenan, ulvan, fucoidan, Glutathione, or a combination thereof. The therapeutically effective amount of one or more phytochemicals comprise a pentacyclic and the one or more complexing agents are selected from whey protein isolate, egg protein isolate, oat protein isolate, hemp protein, sunflower protein isolate pea protein isolate, soybean protein isolate, fishmeal, flaxseed, brown rice protein isolate, N-acetylglucosamine, glucosamine sulfate or N-acetylgalactosamine, glucuronic acid, iduronic acid, galactose chondroitin and glucosamine, glycosaminoglycan, Cysteine, N-Acetyl cysteine, Methionine, DL methionine, L methionine, Tyrosine, taurine, Glycose aminoglycans, mucopolysaccharides, polysaccharide, chondroitin, Chondroitin sulfate, glucosamine, Glucosamine sulfate, carrageenan, ulvan, fucoidan, Glutathione, or a combination thereof. The therapeutically effective amount of one or more phytochemicals comprise a Boswellic isolate and the one or more complexing agents are selected from whey protein isolate, egg protein isolate, oat protein isolate, hemp protein, sunflower protein isolate pea protein isolate, soybean protein isolate, fishmeal, flaxseed, brown rice protein isolate, N-acetylglucosamine, glucosamine sulfate or N-acetylgalactosamine, glucuronic acid, iduronic acid, galactose chondroitin and glucosamine, glycosaminoglycan, Cysteine, N-Acetyl cysteine, Methionine, DL methionine, L methionine, Tyrosine, taurine, Glycose aminoglycans, mucopolysaccharides, polysaccharide, chondroitin, Chondroitin sulfate, glucosamine, Glucosamine sulfate, carrageenan, ulvan, fucoidan, Glutathione, or a combination thereof.

The present invention provides a nutraceutical composition comprising a therapeutically effective amount of one or more phytochemicals selected from a terpenoid; and one or more complexing agents conjugated to a therapeutically effective amount of one or more phytochemicals, wherein the one or more complexing agents are selected from proteins, peptides, amino acids, polysaccharides, sulfated polysaccharides, disaccharides, sulfated disaccharides, monosaccharides, sulfated monosaccharides, amino sugars, glycosaminoglycans, glycol-proteins disposed in a pharmaceutically acceptable excipient, diluent, or carrier.

The present invention provides a method of treating a subject suffering from a phytochemical-related disorder comprising the steps of: identifying a subject in need of treatment of a phytochemical-related disorder; and administering to the subject a nutraceutical composition comprising a phytochemical-acid complex comprising a therapeutically effective amount of one or more phytochemicals selected from a terpenoid; and one or more complexing agents conjugated to a therapeutically effective amount of one or more phytochemicals, wherein the one or more complexing agents are selected from proteins, peptides, amino acids, sulfated polysaccharides, disaccharides, sulfated disaccharides, monosaccharides, sulfated monosaccharides, amino sugars, glycosaminoglycans, glycol-proteins disposed in a pharmaceutically acceptable excipient, diluent, or carrier.

The present invention provides a phytochemical complex comprising a therapeutically effective amount of boswellic acid; and one or more complexing agents conjugated to the therapeutically effective amount of boswellic acid, wherein the one or more complexing agents are selected from proteins, peptides, amino acids, polysaccharides, disaccharides, monosaccharides, amino sugars, glycosaminoglycans, and glycol-proteins, disposed in a pharmaceutically acceptable excipient, diluent, or carrier. In one embodiment the therapeutically effective amount of boswellic acid are non-covalently conjugated to the complexing agent. In one embodiment the therapeutically effective amount of one or more phytochemicals comprise 2, 3, 4, 5, 6, or more phytochemicals. In one embodiment the proteins are selected from Whey protein isolate, Egg protein isolate, Oat protein isolate, Hemp protein, Sunflower protein isolate Pea protein isolate, soybean protein isolate, fishmeal, flaxseed and Brown rice protein isolate. In one embodiment the one or more complexing agents comprise N-acetylglucosamine, glucosamine sulfate or N-acetylgalactosamine, glucuronic acid, iduronic acid, galactose chondroitin and glucosamine, glycosaminoglycan. In one embodiment the one or more complexing agents comprise Cysteine, N-Acetyl cysteine, Methionine, DL methionine, L methionine, Tyrosine, taurine. In one embodiment the one or more complexing agents comprise Glutathione. In one embodiment the therapeutically effective amount of one or more phytochemicals comprise boswellic acid and the one or more complexing agents are selected from whey protein isolate, egg protein isolate, oat protein isolate, hemp protein, sunflower protein isolate pea protein isolate, soybean protein isolate, fishmeal, flaxseed, brown rice protein isolate, N-acetylglucosamine, glucosamine sulfate or N-acetylgalactosamine, glucuronic acid, iduronic acid, galactose chondroitin and glucosamine, glycosaminoglycan, Cysteine, N-Acetyl cysteine, Methionine, DL methionine, L methionine, Tyrosine, taurine, Glycose aminoglycans, mucopolysaccharides, polysaccharide, Chondroitin sulfate and Glucosamine sulfate, Glutathione, or a combination thereof.

The present invention provides a nutraceutical composition comprising a therapeutically effective amount boswellic acid; and one or more complexing agents conjugated to the therapeutically effective amount of boswellic acid, wherein the one or more complexing agents are selected from proteins, peptides, amino acids, polysaccharides, disaccharides, monosaccharides, amino sugars, glycosaminoglycans, glycol-proteins disposed in a pharmaceutically acceptable excipient, diluent, or carrier. In one embodiment the therapeutically effective amount of boswellic acid are non-covalently conjugated to the complexing agent. In one embodiment the therapeutically effective amount of one or more phytochemicals comprise 2, 3, 4, 5, 6, or more phytochemicals. In one embodiment the proteins are selected from Whey protein isolate, Egg protein isolate, Oat protein isolate, Hemp protein, Sunflower protein isolate Pea protein isolate, soybean protein isolate, fishmeal, flaxseed and Brown rice protein isolate. In one embodiment the one or more complexing agents comprise N-acetylglucosamine, glucosamine sulfate or N-acetylgalactosamine, glucuronic acid, iduronic acid, galactose chondroitin and glucosamine, glycosaminoglycan. In one embodiment the one or more complexing agents comprise Cysteine, N-Acetyl cysteine, Methionine, DL methionine, L methionine, Tyrosine, taurine. In one embodiment the one or more complexing agents comprise Glutathione. In one embodiment the therapeutically effective amount of one or more phytochemicals comprise boswellic acid and the one or more complexing agents are selected from whey protein isolate, egg protein isolate, oat protein isolate, hemp protein, sunflower protein isolate pea protein isolate, soybean protein isolate, fishmeal, flaxseed, brown rice protein isolate, N-acetylglucosamine, glucosamine sulfate or N-acetylgalactosamine, glucuronic acid, iduronic acid, galactose chondroitin and glucosamine, glycosaminoglycan, Cysteine, N-Acetyl cysteine, Methionine, DL methionine, L methionine, Tyrosine, taurine, Glycose aminoglycans, mucopolysaccharides, polysaccharide, Chondroitin sulfate and Glucosamine sulfate, Glutathione, or a combination thereof.

The present invention provides a medicament for treating a phytochemical-related disorder comprising: a phytochemical-acid complex comprising a therapeutically effective amount boswellic acid; and one or more complexing agents conjugated to the therapeutically effective amount of boswellic acid, wherein the one or more complexing agents are selected from proteins, peptides, amino acids, polysaccharides, disaccharides, monosaccharides, amino sugars, glycosaminoglycans, glycol-proteins disposed in a pharmaceutically acceptable excipient, diluent, or carrier. In one embodiment the therapeutically effective amount of boswellic acid are non-covalently conjugated to the complexing agent. In one embodiment the therapeutically effective amount of one or more phytochemicals comprise 2, 3, 4, 5, 6, or more phytochemicals. In one embodiment the proteins are selected from Whey protein isolate, Egg protein isolate, Oat protein isolate, Hemp protein, Sunflower protein isolate Pea protein isolate, soybean protein isolate, fishmeal, flaxseed and Brown rice protein isolate. In one embodiment the one or more complexing agents comprise N-acetylglucosamine, glucosamine sulfate or N-acetylgalactosamine, glucuronic acid, iduronic acid, galactose chondroitin and glucosamine, glycosaminoglycan. In one embodiment the one or more complexing agents comprise Cysteine, N-Acetyl cysteine, Methionine, DL methionine, L methionine, Tyrosine, taurine. In one embodiment the one or more complexing agents comprise Glutathione. In one embodiment the therapeutically effective amount of one or more phytochemicals comprise boswellic acid and the one or more complexing agents are selected from whey protein isolate, egg protein isolate, oat protein isolate, hemp protein, sunflower protein isolate pea protein isolate, soybean protein isolate, fishmeal, flaxseed, brown rice protein isolate, N-acetylglucosamine, glucosamine sulfate or N-acetylgalactosamine, glucuronic acid, iduronic acid, galactose chondroitin and glucosamine, glycosaminoglycan, Cysteine, N-Acetyl cysteine, Methionine, DL methionine, L methionine, Tyrosine, taurine, Glycose aminoglycans, mucopolysaccharides, polysaccharide, Chondroitin sulfate and Glucosamine sulfate, Glutathione, or a combination thereof.

### DESCRIPTION OF THE EMBODIMENTS

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

As used herein the term “Conjugation” follows any Conjugation methodology known to the skilled artisan but generally includes the phytochemical being solubilized with a solvent (ethanol, methanol, etc.) under heat ˜500 C, pressure, proper pH (depending on phytochemical) and protected from light while mixing/solubilizing and the mixture is cooled to warmed temperatures (37-450 C). The conjugate material (proteins, polysaccharides, etc.) is added and allowed to mix for a period of time. A vacuum is created to lower boiling point and vaporizing the solvent for removal and drying of the material. In some instances, it is possible to combine two phytochemicals with a conjugate material, e.g., terpenoid and polyphenol can be mixed and conjugated with polysaccharide, or glucosamine sulfate or chondroitin sulfate or a peptide or an amino acid or a protein; or in another embodiment, a pentacyclic triterpene (boswellic acid) can be mixed and conjugated with polysaccharide, or glucosamine sulfate or chondroitin sulfate or a peptide or an amino acid or a protein; or in another embodiment, a pentacyclic triterpene (betulinic acid) can be mixed and conjugated with polysaccharide, or glucosamine sulfate or chondroitin sulfate or a peptide or an amino acid or a protein; or in another embodiment, a monoterpene, (harpagoside) can be mixed and conjugated with polysaccharide, or glucosamine sulfate or chondroitin sulfate or a peptide or an amino acid or a protein.

Terpenoid derivatives include, but not limited to, Boswellia, Boswellia serrata, Boswellia sacra, Boswellia frereana, Boswellia spp., Boswellia extract, Boswellia serrata extract, Boswellia sacra extract, Boswellia frereana extract, Boswellia spp. Extract, Devil's Claw, Devil's Claw extract, Harpagophytum procumbens, Harpagophytum zeyheri, Harpagophytum spp., Harpagophytum procumbens extract, Harpagophytum zeyheri extract, Harpagophytum spp. Extract, monoterpenes (harpagoside), diterpenes, triterpenes, tetracyclic triterpenes, pentacyclic triterpenes (boswellic acid, β-boswellic acid, acetyl-β-boswellic acid, 11-keto-β-boswellic acid, acetyl-11-keto-β-boswellic acid), Chaga, Chaga extract, Inonotus obliquus, Inonotus spp. Inonotux obliquus extract, Inonotus spp. Extract, betulinic acid, and betulin.

Natural sources of terpenoid derivatives include fruits, vegetables, plants, and mushrooms.

As used herein the term “phytochemical” denotes a structural class of mainly natural, but also synthetic or semisynthetic, organic chemicals characterized by the presence of multiples of phenol structural units. The number and characteristics of these phenol structures underlie the unique physical, chemical, and biological properties (e.g., metabolic, toxic, therapeutic, etc.). Examples include (but not limited to) boswellic acid, harpgoside, and betulinic acid. The general physical properties include water-insoluble, moderately water-insoluble and moderately water-soluble compounds. Examples of phytochemical include but are not limited to and include derivatives thereof: terpenoids, polyphenols, phytosterols, flavonols, and . . . .

As used herein the term “proteins” denotes large biomolecules, or macromolecules, consisting of one or more long chains of amino acid residues and includes natural and synthetic and modified R groups to achieve natural, synthetic or modified amino acids. Proteins include Whey protein isolate, Egg protein isolate, Oat protein isolate, Hemp protein, Sunflower protein isolate and Brown rice protein isolate, Other proteins (variable conjugations), Pea protein isolate, soybean protein isolate, fishmeal & flaxseed. Amino acids include Cysteine & N-Acetyl cysteine, Methionine, DL methionine, L methionine, Tyrosine (no conjugation), taurine and the like. N-Acetyl-L-cysteine is the N-acetyl derivative of cysteine. It is used as a mucolytic agent to reduce the viscosity of mucous secretions. It has also been shown to have antiviral effects in patients with HIV due to inhibition of viral stimulation by reactive oxygen intermediates. Methionine is one of nine essential amino acids in humans (provided by food), Methionine is required for growth and tissue repair. A sulphur-containing amino acid, methionine improves the tone and pliability of skin, hair, and strengthens nails. Involved in many detoxifying processes, sulphur provided by methionine protects cells from pollutants, slows cell aging, and is essential for absorption and bio-availability of selenium and zinc. Methionine chelates heavy metals, such as lead and mercury, aiding their excretion. It also acts as a lipotropic agent and prevents excess fat buildup in the liver.

As used herein the term “peptides” denotes small biomolecules, or macromolecules, consisting of one or more short chains of amino acid residues. The term “peptide” in the context of a “peptide compound” or a “peptide complex” is meant as a compound having at least two amino acids linked together by a peptide bond. In some embodiments, the peptide is an oligopeptide, for example a bipeptide, having two amino acids, a tripeptide, having three amino acids, a 4-mer, 5-mer, and the like. In some embodiments, the peptide is an oligopeptide comprises between 2-20 amino acids. In other embodiments, Glutathione is a tripeptide comprised of three amino acids (cysteine, glutamic acid, and glycine) present in most mammalian tissue. Glutathione acts as an antioxidant, a free radical scavenger and a detoxifying agent. Glutathione is also important as a cofactor for the enzyme glutathione peroxidase, in the uptake of amino acids, and in the synthesis of leukotrienes. As a substrate for glutathione S-transferase, this agent reacts with a number of harmful chemical species, such as halides, epoxides and free radicals, to form harmless inactive products. In erythrocytes, these reactions prevent oxidative damage through the reduction of methemoglobin and peroxides. Glutathione is also involved in the formation and maintenance of disulfide bonds in proteins and in the transport of amino acids across cell membranes.

As used herein the term “carrier” denotes a chemical compound that facilitates the incorporation of a compound into cells or tissues. For example, dimethyl sulfoxide (DMSO) is a commonly utilized carrier as it facilitates the uptake of many organic compounds into the cells or tissues of an organism. A common carrier is water, where an aqueous solution of the product of interest is prepared and administered to a subject.

As used herein the term “diluent” denotes chemical compounds diluted in water that will dissolve the compound of interest as well as stabilize the biologically active form of the compound. Salts dissolved in buffered solutions are utilized as diluents in the art. One commonly used buffered solution is phosphate buffered saline because it mimics the salt conditions of human blood. Since buffer salts can control the pH of a solution at low concentrations, a buffered diluent rarely modifies the biological activity of a compound.

In certain embodiments, the same substance can act as a carrier, diluent, or excipient, or have any of the two roles, or have all three roles. Thus, a single additive to the pharmaceutical composition can have multiple functions.

As used herein the term “physiologically acceptable” denotes a carrier or diluent that does not abrogate the biological activity and properties of the compound.

As used herein the term “subject” denotes an animal, preferably a mammal, and most preferably a human, who is the object of treatment, observation or experiment. The mammal may be selected from the group consisting of mice, rats, rabbits, guinea pigs, dogs, cats, sheep, goats, cows, pigs, horses, primates, such as monkeys, chimpanzees, and apes, and humans. Other animals include wildlife (deer, elk, moose, bear, lion, rhinoceros, elephant, etc.), avian (birds, poultry, chicken, turkey, duck, etc.), reptiles (snake, turtle, tortoise, lizard, etc.) and fish (freshwater, saltwater, etc.).

As used herein the term “therapeutically effective amount” denotes an amount of the phytochemical complexed with a protein, peptide, amino acid, polysaccharide, disaccharide or monosaccharide that elicits the biological or medicinal response indicated. This response may occur in a tissue, system, animal or human that is being sought by a researcher, veterinarian, medical doctor or other clinician, and includes alleviation of the symptoms of the disease being treated.

As used herein the term “treat,” “treating,” “treatment,” or any other variation thereof, does not indicate the complete cure from a disorder. Any amelioration of alleviation of the symptoms of a diseases or disorder to any degree, or any increase in the comfort of the subject, is considered treatment.

As used herein the term “Glycosaminoglycans” denotes (GAGs) or mucopolysaccharides are long unbranched polysaccharides consisting of a repeating disaccharide unit. The repeating unit consists of an amino sugar (N-acetylglucosamine, glucosamine sulfate or N-acetylgalactosamine) along with an uronic sugar (glucuronic acid or iduronic acid) or galactose. For example, chondroitin and glucosamine. Chondroitin sulfate is a sulfated glycosaminoglycan (GAG) composed of a chain of alternating sugars (N-acetylgalactosamine and glucuronic acid). It is usually found attached to proteins as part of a proteoglycan. A chondroitin chain can have over 100 individual sugars, each of which can be sulfated in variable positions and quantities. Chondroitin sulfate is an important structural component of cartilage and provides much of its resistance to compression. Glucosamine is commonly used as a treatment for osteoarthritis. It is an amino sugar and a prominent precursor in the biochemical synthesis of glycosylated proteins and lipids. Since glucosamine is a precursor for glycosaminoglycans, and glycosaminoglycans are a major component of joint cartilage, supplemental glucosamine may help to rebuild cartilage and treat arthritis. Other polysaccharides include fucoidan (sulfated polysaccharide) obtained from brown algae and brown seaweed.

As used herein the term “boswellic acid” denotes a triterpenoid found in Boswellia spp. Widely distributed in nature and tropical regions of the world. It is the pentacyclic form of a number of other terpenoids, such as 11arbopo and cannabinoids. Terpenoids include a large number and diverse class of naturally occurring organic phytochemicals derived from the 5-carbon compound isoprene. Terpenes and terpenoids are isoprene polymers. The four major forms of boswellic acid are β-boswellic acid (BA), acetyl-β-boswellic acid (ABA), 11-keto-β-boswellic acid (KBA), and acetyl-11-keto-β-boswellic acid (AKBA). Boswellic acids comprise an estimated 30% of the resins in Boswellia spp. (e.g. Boswellia serrata). These resins contain numerous quantities of other terpenoid compounds. Their interactions, when administered as an extract, are not clearly understood. Boswellic acid is a pentacyclic triterpenoid with potential chemopreventive activity. AKBA has demonstrated activity for increase apoptosis of cancer cells of brain tumors and colon cancer. ABA exhibits anti-inflammatory activity inhibiting the enzyme 5-lipoxygenase (5 LOX).

The present inventors have discovered that the ingestion of a phytochemical-protein complex, or phytochemical-peptide complex or phytochemical-amino acid complex or phytochemical-polysaccharide complex or phytochemical-disaccharide or phytochemical-monosaccharide complex significantly increases solubility and the serum bioavailability of the phytochemical as compared to the ingestion of uncomplexed phytochemical.

Thus, in one aspect, disclosed herein are phytochemical-protein complex, or phytochemical-peptide complex or phytochemical-amino acid complex or phytochemical-polysaccharide complex or phytochemical-disaccharide or phytochemical-monosaccharide complex comprising a phytochemical compound linked to a protein compound or peptide compound or an amino acid or a polysaccharide compound or a disaccharide compound or a monosaccharide. In other embodiments, the peptide compound is a protein or a protein fragment. In some embodiments, a protein is naturally occurring and is a full sequence polypeptide expressed by a cell. In other embodiments, a protein is a synthetic protein having a sequence that is not found in nature. In some embodiments, the synthetic protein is expressed by a cell using recombinant technologies, whereas in other embodiments, the synthetic protein is synthesized using a peptide synthesizer. A protein fragment is an oligo- or polypeptide having a sequence identical to a sequence fragment found in a protein.

In some embodiments, the phytochemical compound is linked covalently to a protein compound or peptide compound or an amino acid or a polysaccharide compound or a disaccharide compound or a monosaccharide. In these embodiments, the phytochemical compound is either bound directly to an amino acid of the peptide, or is bound through a linker compound. In some embodiments, the linker is an alkyl, alkenyl, or alkenyl moiety, which may be substituted with a substituent selected from the group consisting of —OH, —SH, —SO, —COOH, —N—C(O)H, —N—C(O)OH, —C(O)NH, and the like. In some embodiments, the linker is bound to the amino acid or the phytochemical compound through a substituent. In other embodiments, the phytochemical compound is linked by hydrogen bonding to the peptide compound to form the complex. In yet other embodiments, the phytochemical compound is linked by electrostatic forces to the protein compound (or peptide compound or an amino acid or a polysaccharide compound or a disaccharide compound or a monosaccharide) to form the complex. In yet other embodiments, the phytochemical compound is linked by lipophilic interactions (e.g., van der Waals forces) to the protein compound (or peptide compound or an amino acid or a polysaccharide compound or a disaccharide compound or a monosaccharide) to form the complex. In some embodiments, the peptide is a full-length protein. In certain embodiments, the protein is one that is found in the serum of a mammal. In other embodiments, the protein is derived from an animal source other than a mammal. In still other embodiment, the protein is derived from plants, such as grains, legumes, fruits, vegetables, and the like.

Examples of oligo- and polypeptides and full-length proteins used in the complexes described herein include, but are not limited to whey protein, tumor necrosis factor (TNF-α); cyclooxygenase (COX) (including COX-1 and COX-2); al-acid glycoprotein (AGP) (also known as orosomucoid); myeloid differentiation protein 2 (MD-2); any one of the group of enzymes called histone acetyl-transferases (HATs), such as p300/CBP; any one of the group of enzymes called histone deacetylases (HDAC); glyoxalase I (GLOI); xanthine oxidase (XO); a proteasome; sarco (endo) plasmic reticulum Ca²⁺ ATPase (SERCA); human immunodeficiency virus type 1 (HIV-1) protease; any one of the DNA methyltransferases (DNMTs), for example DNMT1; DNA polymerase (pol) any one of the ribonucleases (Rnases), for example Rnase A; any one of the lipoxygenases (LOXs); any one of the matrix metalloproteinases (MMPs); lysozyme; any one of the protein kinase C (PKC) family of enzymes; cellular sarcoma (c-Src); glycogen synthase kinase (GSK)-3β; ErbB2; phosphorylase kinase; any one of the protein reductases, for example thioredoxin reductase (TrxR) and aldose reductase (ALR2); thioredoxin reductase; any one of the caseins; human serum albumin (I); bovine serum albumin (BSA); fibrinogen; β-lactoglobulin (β-LG); α-lactalbumin; human serum immunoglobulin (Ig); FtsZ; transthyretin (TTR); glutathione (GSH); and Kelch-like ECH-associated protein 1 (Keap1).

In some embodiments, the phytochemical-protein complex is a complex of phytochemical and whey protein isolate or a brown rice protein isolate. In certain embodiments the phytochemical is a boswellic acid. In certain embodiments, the whey protein is a milk-derived whey protein or the brown rice protein is a plant derived protein. Milk whey protein is a mixture of β-lactoglobulin (˜65%), α-lactalbumin (˜25%), bovine serum albumin (˜8%), and immunoglobulins. In some of these embodiments, the complex is formed by mixing the phytochemical and the whey protein isolate in ethanol. Thus, in these embodiments, there is no covalent linkage between the phytochemical and the whey protein. In certain embodiments, the ratio of phytochemical to whey protein or brown rice protein is 1:20 w/w. In other embodiments the ratio of a phytochemical to whey protein or brown rice protein is 1:≥40 and 1:≤50 w/w or phytochemical to whey protein or brown rice protein in any increment between 1:>10 and 1:<40. In other embodiments the ratio of phytochemical to whey protein or brown rice protein is 1:50 w/w. In some embodiments, the whey protein is obtained from a commercially available source, which comprises 85-90% whey protein in the available powder. In some embodiments, the brown rice protein is obtained from a commercially available source, which comprises 80-90% brown rice protein in the available powder. In some embodiments, the phytochemical is a boswellic acid and is obtained from a commercially available source.

In some embodiments, the phytochemical-protein complex is a complex of phytochemical and sunflower protein or oat protein. In certain embodiments the phytochemical is a boswellic acid. In certain embodiments, the sunflower protein is a plant derived protein or the oat protein is a plant derived protein. In some of these embodiments, the complex is formed by mixing the phytochemical and the sunflower protein or the oat protein in ethanol. Thus, in these embodiments, there is no covalent linkage between the phytochemical and the sunflower protein or the oat protein. In certain embodiments, the ratio of phytochemical to sunflower protein or oat protein is 1:20 w/w. In other embodiments the ratio of phytochemical to sunflower protein or oat protein is 1:≥40 and 1:≤50 w/w or phytochemical to sunflower protein or oat protein in any increment between 1:>20 and 1:<40. In some embodiments, the sunflower protein is obtained from a commercially available source, which comprises 60-70% sunflower protein in the available powder. In some embodiments, the oat protein is obtained from a commercially available source, which comprises 60-70% oat protein in the available powder. In some embodiments, the phytochemical is a boswellic acid and is obtained from a natural and or commercially available source.

In some embodiments, the phytochemical-protein complex is a complex of phytochemical and hemp protein or flaxseed protein. In certain embodiments the phytochemical is a boswellic acid. In certain embodiments, the hemp protein is a plant derived protein or the flaxseed protein is a plant derived protein. In some of these embodiments, the complex is formed by mixing the phytochemical and the hemp protein or the flaxseed protein in ethanol. Thus, in these embodiments, there is no covalent linkage between the phytochemical and the hemp protein (isolate) or the flaxseed protein (isolate). In certain embodiments, the ratio of phytochemical to hemp protein or flaxseed protein is 1:<100 w/w. In other embodiments the ratio of a phytochemical to hemp protein or flaxseed protein is 1:≥50 and 1:≤100 w/w or phytochemical to hemp protein or flaxseed protein in any increment between 1:>40 and 1:<50. In some embodiments, the hemp protein is obtained from a commercially available source, which comprises 60-70% hemp protein in the available powder. In some embodiments, the flaxseed protein is obtained from a commercially available source, which comprises 60-70% flaxseed protein in the available powder. In some embodiments, the phytochemical is a boswellic acid, and is obtained from a commercially available source.

In another aspect, disclosed herein is a nutraceutical composition comprising a phytochemical-peptide complex, as described herein, and a pharmaceutically acceptable carrier, diluent, or excipient. In certain embodiments the phytochemical is a boswellic acid. In certain embodiments the peptide is a tripeptide. In other embodiments the peptide is glutathione. In some embodiments the glutathione which comprises the three amino acids L-cysteine, L-glutamic acid and glycine is obtained from a commercially available source. The sulfhydryl group of cysteine is primarily responsible for the biological activity of glutathione. As an important antioxidant, glutathione can decrease intra-cellular damage caused by ROS (reactive oxidative species). In some of these embodiments, the complex is formed by mixing the phytochemical and the glutathione in ethanol. Thus, in these embodiments, there is no covalent linkage between the phytochemical and the glutathione. In certain embodiments, the ratio of phytochemical to glutathione is 1:<10 w/w. In other embodiments the ratio of a phytochemical glutathione is 1:≥10 and 1:≤20 w/w or phytochemical to glutathione in any increment between 1:≥1 and 1:≤20.

In another aspect, disclosed herein is a nutraceutical composition comprising a phytochemical-amino acid complex, as described herein, and a pharmaceutically acceptable carrier, diluent, or excipient. In certain embodiments the phytochemical is a boswellic acid. In certain embodiments the amino acid is cysteine. In other embodiments the cysteine amino acid is N-Acetyl-cysteine and is available from a commercially available source. N-acetyl cysteine is a white crystalline powder with a slight odor and sour taste. N-acetyl-cysteine is soluble in water and ethanol. N-acetyl-cysteine molecular formula is C₅H₉NO₃S and a molecular weight of 163.191 g/mol. The sulfhydryl group of cysteine is primarily responsible for the biological activity of N-acetyl-cysteine. In some embodiments the amino acid is methionine. In other embodiments the methionine is DL-methionine or N-acetyl-DL-methionine or L-methionine or D-methionine and is available from a commercially available source. The molecular formula for DL-methionine is C₄H₁₁NO₂S and the molecular weight is 149.208 g/mol. Methionine is an essential amino acid required for growth and tissue repair. In some of these embodiments, the complex is formed by mixing the phytochemical and the amino acid, n-acetyl-cysteine or DL-methionine, in ethanol. Thus, in these embodiments, there is no covalent linkage between the phytochemical and the amino acid n-acetyl-cysteine or DL-methionine. In certain embodiments, the ratio of phytochemical to N-acetyl-cysteine or DL-methionine is 1:1, 1:2, 1:4 or 1:≤10 w/w. In other embodiments the ratio of a phytochemical to N-acetyl-cysteine or DL-methionine is 1:≥10 and 1:≤20 w/w or phytochemical to N-acetyl-cysteine or DL-methionine in any increment between 1:≥1 and 1:≤20.

In other embodiments, disclosed herein include a nutraceutical composition comprising a phytochemical-disaccharide complex, as described herein, and a pharmaceutically acceptable carrier, diluent, or excipient. In certain embodiments the phytochemical is a boswellic acid. In certain embodiments the disaccharide is chondroitin. In other embodiments chondroitin is chondroitin sulfate and is an animal or plant derived mucopolysaccharide or glycosaminoglycan and is available from a commercially available source. Chondroitin sulfate is a white powder and soluble in water and ethanol. Chondroitin sulfate molecular formula is C₁₃H₂₁NO₁₅S and a molecular weight of 463.363 g/mol. In some of these embodiments, the complex is formed by mixing the phytochemical and the chondroitin sulfate in ethanol. Thus, in these embodiments, there is no covalent linkage between the phytochemical and chondroitin sulfate. In certain embodiments, the ratio of phytochemical to chondroitin sulfate is 1:1, 1:2, 1:4 or 1:≤10 w/w. In other embodiments the ratio of a phytochemical to chondroitin sulfate is 1:≥10 and 1:≤20 w/w or the ratio of a phytochemical to chondroitin sulfate in any increment between 1:≥1 and 1:≤20.

In other embodiments, disclosed herein include a nutraceutical composition comprising a phytochemical-monosaccharide complex, as described herein, and a pharmaceutically acceptable carrier, diluent, or excipient. In certain embodiments the phytochemical is a boswellic acid. In certain embodiments the monosaccharide is glucosamine. In other embodiments glucosamine is glucosamine sulfate and is an animal or plant derived monosaccharide and is available from a commercially available source. Glucosamine sulfate is a white powder and soluble in water and ethanol. Glucosamine sulfate molecular formula is C₁₃H₂₁NO₁₅S and a molecular weight of 463.363 g/mol. In some of these embodiments, the complex is formed by mixing the phytochemical and the glucosamine sulfate in ethanol. Thus, in these embodiments, there is no covalent linkage between the phytochemical and glucosamine sulfate. In certain embodiments, the ratio of phytochemical to glucosamine sulfate is 1:1, 1:2, 1:4 or 1:≤10 w/w. In other embodiments the ratio of a phytochemical to glucosamine sulfate is 1:≥10 and 1:≤20 w/w or the ration of a phytochemical to glucosamine sulfate in any increment between 1:≥1 and 1:≤20.

In another aspect, disclosed herein is a nutraceutical composition comprising a phytochemical complexed with a protein, peptide, amino acid, polysaccharide, disaccharide or monosaccharide, as described herein, and a pharmaceutically acceptable carrier, diluent, or excipient. The nutraceutical compositions disclosed herein may be manufactured in a manner that is itself known, e.g., by means of conventional mixing, dissolving, granulating, dragee-making, levigating, emulsifying, encapsulating, entrapping or tableting processes. Nutraceutical compositions disclosed herein thus may be formulated in conventional manner using one or more physiologically acceptable carriers comprising excipients and auxiliaries which facilitate processing of the phytochemical-peptide complexed with a protein, peptide, amino acid, polysaccharide, disaccharide or monosaccharide into preparations which can be used nutraceutically or as a food ingredient (e.g., drink mixes, chocolate, gummies, granola, soup mixes, etc.). Any of the well-known techniques, carriers, and excipients may be used as suitable and as understood in the art; e.g., in Remington's Pharmaceutical Sciences, above.

For oral administration, the phytochemical complexed with a protein, peptide, amino acid, polysaccharide, disaccharide or monosaccharide can be formulated readily by combining the phytochemical complexed with a protein, peptide, amino acid, polysaccharide, disaccharide or monosaccharide with pharmaceutically acceptable carriers well known in the art. Such carriers enable the presently disclosed complexes to be formulated as tablets, pills, dragees, capsules, liquids, gels, syrups, slurries, suspensions and the like, for oral ingestion by a subject. Nutraceutical preparations for oral use can be obtained by mixing one or more solid excipient with the disclosed phytochemical-peptide complexes, optionally grinding the resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinylpyrrolidone, agar, or alginic acid or a salt thereof such as sodium alginate.

Dragee cores are provided with suitable coatings. For this purpose, concentrated sugar solutions may be used, which may optionally contain gum 16arbop, talc, polyvinyl pyrrolidone, 16arbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Nutraceutical preparations that can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for such administration.

Nutraceutical compositions suitable for use in the methods disclosed herein include compositions where the phytochemical-peptide complex is contained in an amount effective to achieve its intended purpose. More specifically, a therapeutically effective amount means an amount of the phytochemical-peptide complex effective to prevent, alleviate or ameliorate symptoms of disease or prolong the survival of the subject being treated.

Typically, the dose range of the phytochemical-complexed with a protein, peptide, amino acid, polysaccharide, disaccharide or monosaccharide administered to the patient is from about 0.5 to 100 mg/kg of the patient's body weight. The dosage may be a single one or a series of two or more given in the course of one or more days, as is needed by the patient. In some embodiments, the dosage is between 0.1 mg to 50 mg. In other embodiments, the dosage is between 1 mg to 10 mg. Other dose ranges include between 10 to 50 mg, between 20 to 50 mg, between 30 to 50 mg, between 40 to 50 mg, between 20 to 40 mg, between 10 to 20 mg, between 10 to 30 mg, between 20 to 30 mg, and between 30 to 40 mg. The dose may also be at 10 mg, 20 mg, 30 mg, 40 mg, 50 mg, 60 mg, 70 mg, 80 mg, 90 mg, 100 mg, 110 mg, 120 mg, 130 mg, 140 mg, 150 mg, 160 mg, 170 mg, 180 mg, 190 mg, or 200 mg. As used above the dosage refers to active agent dosage and cam be in up to 100 mg or 150 mg. In some embodiments, the dosage is 600 mg s.i.d. (once a day). In another embodiment, the phytochemical-complexed with a protein, peptide amino acid, polysaccharide, disaccharide or monosaccharide when given orally, the total dosage is 600-1200 mg per os b.i.d (twice a day) or 600-1200 mg per os t.i.d. (three times a day) or 600-1200 mg per os or 600-1200 mg per os q.i.d. (four times a day)

In another aspect, disclosed herein is a method of treating a disorder, the method comprising identifying a subject in need thereof and administering to the subject a therapeutically effect amount of a phytochemical complex as disclosed herein.

In another aspect, disclosed herein is a method of treating a disorder, the method comprising identifying a subject in need thereof and administering to the subject a therapeutically effect amount of a phytochemical complexed with a protein, peptide, amino acid, polysaccharide, disaccharide or monosaccharide as disclosed herein, where subsequent to the administration, the serum Cmax of phytochemical is >1 ng/ml<2,000 ng/mL. In some embodiments, the serum Cmax of phytochemical is <0.001% of the administered dose of phytochemical. The definition of the pharmacokinetic parameter Cₘₐₓ is well-known to those of skill in the art. Briefly, Cₘₐₓ is the maximum observed plasma concentration after a dosage administration.

In another aspect, disclosed herein is a method of preparing a phytochemical complexed with a protein, peptide, amino acid, polysaccharide, disaccharide or monosaccharide, as described above, the method comprising obtaining a phytochemical; obtaining a protein; obtaining a peptide; obtaining an amino acid; obtaining a polysaccharide; obtaining a disaccharide; or obtaining a monosaccharide and mixing the phytochemical and the protein or the peptide or the amino acid or the polysaccharide or the disaccharide or the monosaccharide in a solvent. In some embodiments, the solvent is a polar solvent, while in other embodiments, the solvent is an apolar solvent. In some embodiments, the polar solvent is water, whereas in other embodiments, the polar solvent is an alcohol. In some embodiments, the alcohol is ethanol or methanol

Example 1: Preparation of phytochemical (in this instance boswellic acid)-Whey Protein Complex. A phytochemical-whey protein complex was prepared for administration to human subjects. The following materials were used: Whey Protein was 90% protein by weight, phytochemical was 95% by weight and 100% ethyl alcohol. Ratio of phytochemical:whey protein of 1:20 w/w. A 0.5% w/v tincture (solution) was prepared by mixing 50 g boswellic acid (from Boswellia serrata extract) powder with 150 mL ethanol. The mixture was placed on a magnetic stirring hot plate, with a speed setting at medium, and temperature setting at 26° C. for 30 minutes or until solution turned clear. To the resulting solution was added 200 grams whey protein isolate powder. The mixture was placed on a rotary evaporator (rotovap) at slow speed (20-30 rpm), having a water bath temperature of 26° C., and low vacuum for 3-4 hours or until the ethanol was evaporated. Alternatively, the mixture was placed in a lyophilizer. The final product was a fine and light yellow colored powder. The powder re-solubilizes in water with stirring. Alternative method—In a vacuum blender the phytochemical is added to a 150 ml. solvent (methanol, acetate, ethanol) at the rate of 50 grams per 150 milliliters. The mixture is blended at 26° C. for 60 minutes or until mixture is clear. 200 grams of whey protein per 150 solvent is added and continued mixing at 26° C. for thirty minutes. Low vacuum is applied to remove solvent until dry. Ratio of a single phytochemical:whey protein of 50 mg phytochemical:1 gm powder or 1:20 w/w. The above procedure was repeated, except with 50 g phytochemical powder and 200 g brown rice protein isolate powder. A similar product was obtained with a ration of 1:10 w/w of phytochemical to brown rice protein. The above procedure was repeated, except with 50 g phytochemical powder and 200 g brown rice protein isolate powder. A similar product was obtained with a ration of 1:20 w/w of phytochemical to brown rice protein. The above procedure was repeated, except with boswellic acid powder and 200 g brown rice protein isolate powder. A similar product was obtained with a ratio of 1:20 w/w of boswellic acid to brown rice protein, boswellic acid-peptide, boswellic acid-amino acid, boswellic acid-chondroitin, boswellic acid-glucosamine, boswellic acid-polysaccharide.

The formulations may include the active agent in communication with a polysaccharide, mucopolysaccharide, glycosaminoglycan, disaccharide, monosaccharide or a amino sugar that is synthetic or naturally occurring. For example, the composition may be boswellic acid-glucosamine conjugates, and boswellic acid-chondroitin conjugates.

The formulations may include the phytochemicals, like boswellic acid conjugates and examples of the formulation include boswellic acid-protein, boswellic acid-peptides, boswellic acid-amino acid, boswellic acid-polysaccharide, boswellic acid-glucosamine or boswellic acid-chondroitin. The process of making these conjugates include solvent assisted blending of conjugates and examples of the formulation includes 10-250 mg boswellic acid per 1 gram peptides, or 1 gram n-acetyl-cysteine or 1 gram DL-methionine or 1 gram chondroitin sulfate or 1 gram glucosamine sulfate. These formulations may be used in animal formulations and human formulations.

In any of the embodiments may include common peptides (proteins) to be used including Proteins like whey, brown rice, egg, hemp protein, flaxseed protein, etc.; Amino acids—cysteine, methionine; saccharides to be used include glucosamine, polysaccharide & chondroitin.

The present invention provides boswellic acid-glucosamine conjugates using glucosamine sulfate which is a white crystalline powder with a MW of 277.2496 g/mol and a chemical formula C₆H₁₅NO₉S and a melting point of 192° C. Glucosamine can be found in marine (shellfish, crustacean) and fermentation of grains (primarily corn or wheat). It is slightly water soluble and provides benefits like joint health. An example of the boswellic acid-glucosamine sulfate complex is prepared using the following materials glucosamine sulfate about 99%, Boswellia serrata extract powder about 65% boswellic acid by weight, ethanol about 95% (ethyl alcohol). The processing or blending includes combining 50 grams boswellic acid with 150 milliliters ethanol is placed in a rotary mixing vessel at room temperature and medium speed (20 rpm) and blended for 30 minutes and 200 grams glucosamine sulfate powder is added with continued mixing for another 30 minutes. A low vacuum is generated in the vessel to remove the ethanol solvent resulting powder is a fine, slightly crystalline, light yellow color and able to solubilize in water. The ratio of boswellic acid:glucosamine sulfate is 1:4 w/w.

TABLE 2
Proteins identified in Test Articles with > 10 spectra

| | | | Number of Assigned | | |
|---|---|---|---|---|---|
| | | | Spectra | | |
| | | | Test | Test | Test |
| | Accession | Molecular | Article | Article | Article |
| Identified Proteins | Number | Weight | 1 | 2 | 3 |
| Major allergen beta- | B5B0D4_ BOVIN | 20 kDa | 357 | 447 | 95 |
| lactoglobulin OS = Bos taurus | | | | | |
| PE = 2 SV = 1 | | | | | |
| Beta-casein OS = Bos taurus | CASB_BOVIN | 25 kDa | 128 | 150 | 37 |
| GN = CSN2 PE = 1 SV = 2 | | | | | |
| Glycosylation-dependent cell | GLCM1_BOVIN | 17 kDa | 108 | 121 | 42 |
| adhesion molecule 1 OS = Bos | | | | | |
| taurus GN = GLYCAM1 PE = 1 | | | | | |
| SV = 2 | | | | | |
| Serum albumin OS = Bos taurus | ALBU_BOVIN | 69 kDa | 51 | 42 | 0 |
| GN = ALB PE = 1 SV = 4 | | | | | |
| Alpha-lactalbumin protein | G9G9X6_BOVIN | 16 kDa | 42 | 43 | 0 |
| variant D OS = Bos taurus | (+1) | | | | |
| GN = LALBA PE = 3 SV = 1 | | | | | |
| Kappa-casein OS = Bos taurus | CASK_BOVIN | 21 kDa | 0 | 40 | 0 |
| GN = CSN3 PE = 1 SV = 1 | | | | | |
| Alpha-S1-casein OS = Bos | CASA1_BOVIN | 25 kDa | 43 | 55 | 0 |
| taurus GN = CSN1S1 PE = 1 | | | | | |
| SV = 2 | | | | | |
| Alpha-S2-casein OS = Bos | CASA2_BOVIN | 26 kDa | 36 | 44 | 0 |
| taurus GN = CSN1S2 PE = 1 | | | | | |
| SV = 2 | | | | | |
| Uncharacterized protein | G3N0V0_BOVIN | 36 kDa | 26 | 25 | 0 |
| (Fragment) OS = Bos taurus | | | | | |
| PE = 1 SV = 1 | | | | | |
| Putative uncharacterized | A5D7Q2_BOVIN | 52 kDa | 14 | 21 | 0 |
| protein OS = Bos taurus PE = 2 | | | | | |
| SV = 1 | | | | | |
| Osteopontin OS = Bos taurus | OSTP_BOVIN | 31 kDa | 15 | 15 | 0 |
| GN = SPP1 PE = 1 SV = 2 | (+1) | | | | |
| Polymeric immunoglobulin | PIGR_BOVIN | 82 kDa | 0 | 15 | 0 |
| receptor OS = Bos taurus | | | | | |
| GN = PIGR PE = 2 SV = 1 | | | | | |
| Alpha-1-acid glycoprotein | AlAG_BOVIN | 23 kDa | 14 | 13 | 0 |
| OS = Bos taurus GN = ORM1 | (+1) | | | | |
| PE = 2 SV = 1 | | | | | |
| Butyrophilin subfamily 1 | BT1A1_BOVIN | 59 kDa | 15 | 16 | 0 |
| member A1 OS = Bos taurus | | | | | |
| GN = BTN1A1 PE = 1 SV = 2 | | | | | |
| Transthyretin OS = Bos taurus | TTHY_BOVIN | 16 kDa | 0 | 11 | 0 |
| GN = TTR PE = 1 SV = 1 | | | | | |

Comparison using Progenesis QI for Proteomics. As the potential modification of peptides with phytochemicals remains undefined, a comparison between Test Articles 1 and 2 was made that is independent of protein identification and peptide assignments. For this comparison, the retention time versus the m/z pattern of each run was compared using Progenesis QI for Proteomics (Demo licence; Non-Linear Dynamics) with the assumption that a phytochemical modification will change the retention time and the mass-to-charge ratio (m/z) of the peptide. An overview of both runs demonstrates that the majority of ‘features’—molecules detected by the mass spectrometer—are very similar between the samples. The software detected over 21,000 features, with over 1,700 showing abundance changes of greater than 500 fold (see report). While many of these changes are due to variations in the alignment between the runs, there are several that may represent peptides that are modified by the phytochemical treated. It is anticipated that the most specific changes would be represented by 1) appearance of a new feature in Test Article 2—representing the modified peptide, and 2) a decrease in intensity of a feature in Test Article 2 compared with Test Article 1—representing the fraction of the material that is modified as it is unlikely that the modification will occur for all peptides.

What is claimed is:

1. A phytochemical complex comprising:
a therapeutically effective amount of boswellic acid; and
one or more complexing agents conjugated to the therapeutically effective amount of boswellic acid, wherein the one or more complexing agents are selected from proteins, peptides, amino acids, polysaccharides, disaccharides, monosaccharides, amino sugars, glycosaminoglycans, and glycol-proteins, disposed in a pharmaceutically acceptable excipient, diluent, or carrier.

2. A nutraceutical composition comprising:
a therapeutically effective amount boswellic acid; and
one or more complexing agents conjugated to the therapeutically effective amount of boswellic acid, wherein the one or more complexing agents are selected from proteins, peptides, amino acids, polysaccharides, disaccharides, monosaccharides, amino sugars, glycosaminoglycans, glycol-proteins disposed in a pharmaceutically acceptable excipient, diluent, or carrier.

3. A medicament for treating a phytochemical-related disorder comprising:
a phytochemical-acid complex comprising a therapeutically effective amount boswellic acid; and one or more complexing agents conjugated to the therapeutically effective amount of boswellic acid, wherein the one or more complexing agents are selected from proteins, peptides, amino acids, polysaccharides, disaccharides, monosaccharides, amino sugars, glycosaminoglycans, glycol-proteins disposed in a pharmaceutically acceptable excipient, diluent, or carrier.

4. The phytochemical complex of claim 1, wherein the therapeutically effective amount of boswellic acid are non-covalently conjugated to the complexing agent.

5. The phytochemical complex of claim 1, wherein the therapeutically effective amount of one or more phytochemicals comprise 2, 3, 4, 5, 6, or more phytochemicals.

6. The phytochemical complex of claim 1, wherein the proteins are selected from whey protein isolate, egg protein isolate, oat protein isolate, hemp protein, sunflower protein isolate pea protein isolate, soybean protein isolate, fishmeal, flaxseed and brown rice protein isolate.

7. The phytochemical complex of claim 1, wherein the one or more complexing agents comprise N-acetylglucosamine, glucosamine sulfate or N-acetylgalactosamine, glucuronic acid, iduronic acid, galactose chondroitin and glucosamine, glycosaminoglycan.

8. The phytochemical complex of claim 1, wherein the one or more complexing agents comprise Cysteine, N-Acetyl cysteine, Methionine, DL methionine, L methionine, Tyrosine, taurine.

9. The phytochemical complex of claim 1, wherein the one or more complexing agents comprise Glutathione.

10. The phytochemical complex of claim 1, wherein the therapeutically effective amount of one or more phytochemicals comprise boswellic acid and the one or more complexing agents are selected from whey protein isolate, egg protein isolate, oat protein isolate, hemp protein, sunflower protein isolate pea protein isolate, soybean protein isolate, fishmeal, flaxseed, brown rice protein isolate, N-acetylglucosamine, glucosamine sulfate or N-acetylgalactosamine, glucuronic acid, iduronic acid, galactose chondroitin and glucosamine, glycosaminoglycan, Cysteine, N-Acetyl cysteine, Methionine, DL methionine, L methionine, Tyrosine, taurine, Glycose aminoglycans, mucopolysaccharides, polysaccharide, Chondroitin sulfate and Glucosamine sulfate, Glutathione, or a combination thereof.
